(19) United States
(12) Patent Application Publication
(10) Pub. No.: US 2008/0247977 A1
(43) Pub. Date: Oct. 9, 2008

(54) Anhydrous Film for Lip Make-Up or Care

(75) Inventors: Jean-Yves Le Gendre, Paris (FR); Vanina Filippi, Paris (FR)

Correspondence Address: OLIFF & BERRIDGE, PLC, P.O. BOX 320850, ALEXANDRIA, VA, 22320-4850, US

(73) Assignee: L'OREAL, Paris (FR)

(21) Appl. No.: 10/585,872

(22) PCT Filed: Jan. 21, 2005
(86) PCT No.: PCT/FR05/50035

§ 371 Date: Oct. 10, 2007

Related U.S. Application Data
(60) Provisional application No. 60/541,238, filed on Feb. 4, 2004.

(30) Foreign Application Priority Data
Jan. 21, 2004 (FR) 0450109

(51) Int. Cl. A61K 8/18; A61Q 1/04

(52) U.S. Cl. 424/64

(57) ABSTRACT

The invention concerns a cosmetic product for lip make-up or care in the form of an anhydrous film comprising: a) at least one water-soluble polymer, and b) at least one dye. Said anhydrous film dissolves in contact with saliva to provide lip make-up or treatment.

A subject matter of the present invention is a cosmetic product for making up and/or caring for the lips comprising an anhydrous film comprising a polymer layer in which is included at least one coloring agent and, if appropriate, at least one active compound.

Various types of products for caring for and making up the lips are currently available. They can be divided schematically between the two following categories:

compositions with a liquid or pasty texture which can be applied with a finger or using devices such as roller-ball applicators, chamfered nozzles or brushes,

and compositions of greater hardness, such as pencils or sticks.

The devices for packaging and applying these compositions involve, from the first use, exposure of the composition to the air. For obvious reasons, this exposure may detrimentally affect the qualities of the composition by oxidation and/or by evaporation of the volatile compounds liable to be formulated therein. In addition, this repeated exposure at each use of the composition presents a problem of hygiene due to the increased risk of contamination of the composition by the external environment.

The present invention is targeted specifically at providing a novel type of cosmetic product for the lips which overcomes the abovementioned disadvantages. In particular, this cosmetic product is a single-use product.

A subject matter of the present invention is a cosmetic product for making up and/or caring for the lips comprising an anhydrous film, this film being formed of at least one layer composed of a matrix based on at least one water-soluble polymer in which is included at least one coloring agent.

In a specific embodiment, the present invention relates to a cosmetic product for making up and/or caring for the lips comprising an anhydrous film formed of at least one layer composed of a matrix based on at least one water-soluble polymer in which is included at least one water-soluble dye and, if appropriate, one water-soluble active compound.

The cosmetic product can advantageously be dissolved by contact with the saliva. The product of the invention exhibits the advantage of quickly dissolving, once applied to the lips. The film is advantageously strong and able to be grasped. It has a soft consistency, with the result that it adopts the shape and the curvature of the lips from its application.

The term “anhydrous” is understood to mean a film having a water content of less than 10%, in particular of less than 5%, especially of less than 3%, and more particularly not comprising water.

Advantageously, the product in accordance with the invention is provided as a film in the form of a square, rectangle or disk with a length of approximately ten centimeters which the user cuts up according to the shape and dimensions of her own lips and according to the result desired.

In another embodiment, the product in accordance with the invention is precut to the average shape and average dimensions of the lips, with the result that it can be applied directly by the user to each of the lips. For reasons of convenience and of hygiene, this option is generally favored. The precut film can have the shape of a single lip. The precut film can also have the shape of two lips joined at their ends, so that the user, on pursing her lips, makes up the two lips at the same time.

In another embodiment, the product of the invention has the shape of a rectangular, square or circular sheet which the user fold in two before pursing it between the two lips.

The cosmetic product can be provided in the form of a strong film with a thickness ranging from 10 to 1000 μm. According to a specific embodiment, this film exhibits a thickness ranging from 20 to 500 μm, in particular from 50 to 250 μm and more particularly from 50 to 200 μm.

This film is formed of at least one layer, the matrix of which is composed of at least one water-soluble polymer, in which is included at least one coloring agent. According to another embodiment, the film is composed of a single layer comprising at least one water-soluble polymer.

According to a specific embodiment, the cosmetic product according to the invention comprises, in addition to the film defined above, a water-insoluble support, the aim of which is to make possible ready application of the film to the lips. This support is in particular flexible. The water-insoluble support can be made of a material chosen from polyurethanes, thermoplastic elastomers of the styrene/butadiene/styrene, styrene/ethylene/butadiene/styrene, ethylene/vinyl acetate or co-ether-ester type, polyethylenes, polypropylenes or silicones. The nature and the form of the support will be appropriately chosen according to the nature of the surface to be treated so as to allow it advantageously to be massaged on the surface to be treated without risk to the latter and with maximum comfort.

Such supports are sold in particular under the trade names: Baydur®, Daltoflex®, Uroflex®, Hyperlast®, Inspire®, Desmopan®, Estane®, Lastane®, Texin®, Cariflex®, Kraton®, Solprene®, Elvax®, Escorene®, Optene®, Arnitel®, Hytrel® or Riteflex®.

Alternatively, the support can be in the form of a nonwoven fabric, in particular of cellulose, of viscose, of cotton or of synthetic fibers.

The thickness of the support ranges in particular from 0.01 mm to 2 mm and more particularly from 0.02 to 0.2 mm.

In particular, the cosmetic product according to the invention can be composed solely of a film as described above and be devoid of a water-insoluble support layer and in particular be devoid of a flexible water-insoluble support.

The water-soluble polymers used in the cosmetic product according to the invention can be of synthetic or natural origin, if appropriate modified by chemical reactions. They can be film-forming.

Mention may in particular be made, by way of illustration of the water-soluble polymers which can be used according to the invention, of the following polymers:

(1) polymers resulting from the copolymerization of a monomer derived from a vinyl compound carrying a carboxyl group, such as more particularly acrylic acid, methacrylic acid, maleic acid or α-chloroacrylic acid, and of a basic monomer derived from a substituted vinyl compound comprising at least one basic atom, such as more particularly a dialkylaminoalkyl methacrylate and acrylate and a dialkylaminoalkylmethacrylamide and -acrylamide. Such compounds are disclosed in U.S. Pat. No. 3,836,537;

(2) polymers comprising units deriving:

a) from at least one monomer chosen from acrylamides or methacrylamides substituted on the nitrogen atom by an alkyl radical,

b) from at least one acidic comonomer comprising one or more reactive carboxyl groups, and

c) from at least one basic comonomer, such as esters, having primary, secondary, tertiary and quaternary amine substituents, of acrylic and methacrylic acids and the quaternization product of dimethylaminoethyl methacrylate with dimethyl or diethyl sulfate;

(3) crosslinked alkylpolyaminoamides derived completely or partially from polyaminoamides;

(4) polymers comprising zwitterionic units;

(5) polymers derived from the N-carboxy-alkylation of chitosan, such as, for example, the N-(carboxymethyl)chitosan or the N-(carboxybutyl)-chitosan sold under the name “Evalsan®” by Jan Dekker; and

(6) (C₁-C₅)alkyl vinyl ether/maleic anhydride copolymers partially modified by semiamidation with an N,N-dialkylaminoalkylamine, such as N,N-dimethylamino-propylamine, or by semiesterification with an N,N-dialkanolamine. These copolymers can also comprise other vinyl comonomers, such as vinylcaprolactam.

Mention may also be made, as other water-soluble polymers which can be used according to the invention, of:

proteins (or polymers of protein type), such as wheat or soybean proteins; keratin, for example keratin hydrolysates and sulfonic keratins; casein; albumin; collagen; glutelin; glucagon; gluten; zein; gelatins and their derivatives;

polymers deriving from chitin or chitosan, such as anionic, cationic, amphoteric or nonionic polymers of chitin or of chitosan;

polymers of polysaccharide type, such as in particular:
cellulose polymers, such as hydroxy-ethylcellulose, hydroxypropylcellulose, hydroxypropyl methylcellulose, methylcellulose, ethylhydroxyethylcellulose, carboxymethylcellulose, and quaternized cellulose derivatives; and
starches and their derivatives;

acrylic polymers or copolymers, such as polyacrylates or polymethacrylates;

vinyl polymers, such as polyvinyl-pyrrolidones, copolymers of methyl vinyl ether and of maleic anhydride, the copolymer of vinyl acetate and of crotonic acid, or copolymers of vinylpyrrolidone and of vinyl acetate;

copolymers of vinylpyrrolidone and of caprolactam; poly(vinyl alcohol)s;

optionally modified polymers of natural origin, such as:
gum arabic, guar gum, xanthan derivatives or karaya gum;
alginates, carrageenans, levans and other algal colloids;
glycoaminoglycans, hyaluronic acid and its derivatives;
shellac, sandarac gum, dammars, elemis or copals;
deoxyribonucleic acid;
mucopolysaccharides, such as hyaluronic acid, chondroitin sulfate, and mixtures of these.

Mention may also be made of caprolactams, pullulan, pectin, mannan and galactomannans, glucomannans and their derivatives.

Of course, the product in accordance with the invention can comprise a mixture of such polymers.

The water-soluble polymer is generally present in a proportion of 5 to 99.99% by weight, in particular of 10 to 95% by weight and more particularly of 20 to 90% by weight, with respect to the total weight of said product.

The cosmetic product according to the invention comprises at least one coloring agent.

The coloring agent can be chosen from water-soluble dyes, pigments and their mixtures.

The coloring agent can represent, for example, from 0.005 to 20% by weight, in particular from 0.01 to 10% by weight and more particularly from 0.05 to 5% by weight, with respect to the total weight of said cosmetic product.

According to a specific embodiment, the coloring agent used in the product in accordance with the invention comprises at least one water-soluble dye.

Mention may be made, among water-soluble dyes which can be used in the compositions according to the invention, for example, of copper sulfate, iron sulfate, water-soluble sulfopolyesters, rhodamines, natural dyes, such as carotene and beetroot juice, methylene blue, caramel, the disodium salt of tartrazine and the disodium salt of fuchsin, and their mixtures.

The cosmetic product can comprise water-insoluble coloring agents, such as pigments, in particular pearlescent agents or glitter.

According to one embodiment, the coloring agent used in the product in accordance with the invention comprises at least one pigment.

The pigments can be inorganic and/or organic, interference, goniochromatic, fluorescent, pearlescent or reflecting or in the form of glitter.

Mention may be made, among inorganic pigments, of titanium dioxide, optionally treated at the surface, zirconium or cerium oxides, and also zinc, iron (black, yellow or red) or chromium oxides, manganese violet, ultramarine blue, chromium hydrate and ferric blue. Mention may be made, among organic pigments, of carbon black, pigments of organic lakes of barium, strontium, calcium or aluminum type, including those subject to certification by the Food and Drug Administration (FDA) (example, D&C or FD&C) and those devoid of the FDA certification, such as lakes based on cochineal carmine.

The pearlescent pigments or pearlescent agents can be chosen from white pearlescent pigments, such as mica covered with titanium oxide or with bismuth oxychloride, colored pearlescent pigments, such as titanium oxide-coated mica with iron oxides, titanium oxide-coated mica with in particular ferric blue or chromium oxide or titanium oxide-coated mica with an organic pigment of the abovementioned type, and pearlescent pigments based on bismuth oxychloride. Mention may be made, among commercially available pearlescent agents, of the “Timica®” and “Flamenco®” pearlescent agents sold by Engelhard and the “Timiron®” pearlescent agents sold by Merck.

Use may also be made of goniochromatic pigments, such as pigments with a multilayer interference structure, for example with the Al/SiO₂/Al/SiO₂/Al structure, sold by DuPont de Nemours; with the Cr/MgF₂/Al/MgF₂/Cr structure, sold under the name “Chromaflair®” by Flex; with the MoS₂/SiO₂/Al/SiO₂/MoS₂, Fe₂O₃/SiO₂/Al/SiO₂/Fe₂O₃ or Fe₂O₃/SiO₂/Fe₂O₃/SiO₂/Fe₂O₃ structure, sold under the name “Sicopearl®” by BASF; with the Mos₂/SiO₂/mica coated with oxide/SiO₂/MoS₂, Fe₂O₃/SiO₂/mica coated with oxide/SiO₂/Fe₂O₃, TiO₂/SiO₂/TiO₂ or TiO₂/Al₂O₃/TiO₂ structure, sold under the name “Xirona®” by Merck (Darmstadt). Mention may also be made of the “Infinite Colors®” pigments from Shiseido.

Use may also be made of reflecting pigments, such as particles comprising a glass substrate coated with silver, in the form of platelets, such as those sold, for example, under the name “Microglass Metashine REFSX 2025 PS®” by Toyal; particles comprising a glass substrate coated with nickel/chromium/molybdenum alloy, such as those sold, for example, under the name “Crystal Star GF 55®”, “GF 2525®” by this same company; pigments of the “Reflecks®” trade mark, sold by Engelhard, comprising a glass substrate coated with brown iron oxide; particles comprising a stack of at least two layers of polymers are, for example, sold by 3M under the name “Mirror Glitter®”.

Use may be made, as goniochromatic liquid crystal particles, for example, of those sold by Chenix and that sold under the name “Helicone® HC” by Wacker.

The cosmetic product according to the invention can be a product for the nontherapeutic care of the lips. Thus, the product according to the invention can therefore comprise, in addition to the coloring agent, at least one cosmetically active compound. Use may be made of any active principle known for having a cosmetic action by topical application.

The active compound can be chosen from compounds having a moisturizing, softening, emollient, healing, regenerating, soothing or antiwrinkle action, a protective action against solar radiation or an action targeted at enhancing blood microcirculation in order to render the lips naturally more colored, in order to increase their volume or in order to smooth their surface.

Mention may be made, among the active compounds, of lipophilic vitamins and their esters, such as vitamin E (tocopherol) and its derivatives, (for example the acetate), vitamin A (retinol) and its derivatives (for example, retinyl palmitate), polyunsaturated fatty acids, essential oils, plant extracts, sphingolipids and ceramides, sunscreens, such as, for example, octyl methoxycinnamate, such as that sold under the name “Parsol MCX®”, 3-benzophenone, such as that sold under the name “Uvinul M40®”, or butyl-methoxydibenzoylmethane, such as that sold under the name “Parsol 1789®”, retinoic acid and its esters, and their mixtures.

The polymer matrix can comprise at least one active compound chosen from the following compounds: α-hydroxy acids, such as lactic acid or glycolic acid, ascorbic acid (vitamin C) and its biologically compatible salts, enzymes, components with a tightening effect, hydroxy acids and their salts, hydroxylated polyacids, sucrose and its derivatives, urea, amino acids, oligopeptides, water-soluble plant extracts, peptide or protein hydrolysates, bacterial ferments, trace elements, hyaluronic acid and its salts, muco-polysaccharides, vitamins B2, B6, H, PP, panthenol, folic acid, β-hydroxy acids, such as acetylsalicylic acid, allantoin, glycyrrhetic acid, kojic acid, hydro-quinone, arginine, lysine, proline, serine, flavonoids, ginkgo biloba extracts, and their mixtures.

According to a specific embodiment, the product in accordance with the invention comprises at least one water-soluble active compound.

The term “water-soluble active compound” is understood to mean an active compound having a solubility in the water, measured at 25° C., at least equal to 0.1 g/l (a solution is obtained which is macroscopically isotropic and transparent, and colored or colorless). This solubility is in particular greater than or equal to 1 g/l.

The product of the invention can comprise emollients, such as polyols, in particular glycerol, sorbitol and other polyols of similar structure, or ethers of pentaerythritol and of polyalkylene glycol. For example, use is made of the ether of penta-erythritol and of polyethylene glycol comprising 5 oxyethylene units (5 OE) (CTFA name: PEG-5 Penta-erythrityl Ether), the ether of pentaerythritol and of polypropylene glycol comprising 5 oxypropylene units (5 OP) (CTFA name: PPG-5 Pentaerythrityl Ether) and their mixtures, more especially the PEG-5 Penta-erythrityl Ether, PPG-5 Pentaerythrityl Ether and soybean oil mixture sold under the name “Lanolide®”, by Vevy, a mixture in which the constituents are found in a 46/46/8 ratio by weight: 46% of PEG-5 Pentaerythrityl Ether, 46% of PPG-5 Pentaerythrityl Ether and 8% of soybean oil.

The cosmetic product according to the invention can also be devoid of active compound. The cosmetic product of the invention is a nontherapeutic cosmetic product.

The cosmetic product according to the invention can be devoid of preservative. This is, of course, advantageous in terms of harmlessness.

The product according to the invention can additionally comprise at least one compound chosen from plasticizers, fibers, whitening agents, surfactants, dispersants, antioxidants, pH-regulating agents, sweeteners, fragrances, flavorings or one of their mixtures. Mention may in particular be made, as plasticizing agent, of glycerol, sorbitol or polyethylene glycol, mono- or disaccharides, dipropylene glycol, butylene glycol or pentylene glycol.

Mention may in particular be made, as surface-active agent, of polysorbates and polyalkyl-siloxanes.

Advantageously, the cosmetic product in accordance with the invention can be used individually and exhibits excellent preservation, including without addition of preservative, in particular by virtue of its dry nature.

The product of the invention can be packaged in an article which facilitates the grasping thereof, such as that disclosed in patent application FR 03/51002, the content of which is included in the present application by reference. The film can in particular be packaged in a dispensing plastic box, in a separate bag or in a blister pack. The product according to the invention can be packaged in a box of the type with a drawer or with a lid hinged on a base. The box can comprise means intended to facilitate the dispensing of the articles. The dispensing means can be of the type of those disclosed, for example, in patents U.S. Pat. No. 2,973,882, GB-A-2 358 627, CH-A-461 025 or U.S. Pat. No. 6,578,732.

Another subject matter of the present invention is a method for making up or caring for the lips, characterized in that it comprises the fact of applying, to the lips, a makeup or care product as defined above. In particular, the method is a method for making up the lips comprising the fact of applying, to the lips, a makeup product as defined above and of dissolving the product by the action of the saliva and the rubbing of the lips against one another or the pressure of the finger.

Advantageously, the method comprises a stage consisting in massaging the lips, if appropriate through the support, so as to promote the spreading of the dissolved film over the surface of the lips.

According to a specific embodiment, the makeup or care method according to the invention is carried out while applying beforehand, to the lips, a known composition for making up and/or caring for the lips, such as, for example, a lipstick, a balm or a gloss.

According to another embodiment of the invention, not to the exclusion of the embodiment presented above, the use of the makeup or care method as defined above is followed by an additional stage in which another composition, such as, for example, a lipstick, a lip balm or a gloss, is applied.

The cosmetic product according to the invention can be prepared according to conventional methods well known to a person skilled in the art. Advantageously, its preparation does not comprise a stage carried out at a temperature of greater than 50° C. The cosmetic product in accordance with the invention can thus optionally comprise compounds which may be sensitive to such temperatures.

The present invention will be illustrated below with the help of nonlimiting examples.

### EXAMPLE 1

### Makeup Film for the Lips

| | |
|---|---|
| Hydroxypropylmethylcellulose | 10 g |
| D-Panthenol | 2 g |
| Glycerol | 5 g |
| Disodium salt of fuchsin | 0.5 g |
| Purified water | 50 g |

The combined ingredients are dissolved in the purified water with stirring. The solution is deposited on silicone-treated paper in a thickness of the order of 500 μm and then dried at a temperature of 50° C. After drying, the film obtained is cut into the form of strips with a width of 5 mm and a length of 40 mm.

### EXAMPLE 2

### Makeup Film for the Lips

| | |
|---|---|
| Hydroxypropylmethylcellulose | 10 g |
| Glycerol | 3 g |
| Titanium dioxide/brown | 1 g |
| iron oxide mixture | |
| Purified water | 50 g |

The film is prepared according to the protocol described in example 1.

1. The use of at least one water-soluble polymer and of at least one coloring agent in the manufacture of a cosmetic product comprising an anhydrous film, this film being formed of at least one layer composed of a matrix based on said water-soluble polymer in which is included said coloring agent, said cosmetic product being intended for making up and/or caring for the lips.

2. The use as claimed in claim 1, characterized in that the polymer is chosen from polymers of protein type; polymers deriving from chitin or from chitosan; polysaccharide polymers; acrylic polymers or copolymers; vinyl polymers; copolymers of vinylpyrrolidone and of caprolactam, and their blends.

3. The use as claimed in claim 1, characterized in that said coloring agent is chosen from water-soluble dyes, pigments and their mixtures.

4. The use as claimed in claim 1, characterized in that the coloring agent comprises at least one water-soluble coloring agent.

5. The use as claimed in claim 4, characterized in that said water-soluble dye is chosen from copper sulfate, iron sulfate, water-soluble sulfopolyesters, rhodamines, natural dyes, such as carotene and beetroot juice, methylene blue, caramel, the disodium salt of tartrazine and the disodium salt of fuchsin, and their mixtures.

6. The use as claimed in claim 1, characterized in that said coloring agent comprises at least one pigment.

7. The use as claimed in claim 6, characterized in that said pigment is chosen from reflecting pigments, goniochromatic pigments, interference pigments, pearlescent pigments, fluorescent pigments, glitter and their mixtures.

8. The use as claimed in claim 1, characterized in that the coloring agent is present in a content ranging from 0.005 to 20% by weight, in particular from 0.01 to 10% by weight and more particularly from 0.05 to 5% by weight, with respect to the total weight of said cosmetic product.

9. The use as claimed in claim 1, characterized in that the product additionally comprises at least one active compound.

10. The use as claimed in claim 9, characterized in that the active compound is water-soluble.

11. The use as claimed in claim 9, characterized in that the active compound is chosen from compounds having a moisturizing, softening, emollient, healing, regenerating, soothing or antiwrinkle action, a protective action against solar radiation or an action targeted at enhancing blood microcirculation in order to render the lips naturally more colored, in order to increase their volume or in order to smooth their surface.

12. The use as claimed in claim 9, characterized in that the active compound is chosen from vitamin E and its derivatives, vitamin A and its derivatives, polyunsaturated fatty acids, essential oils, plant extracts, sphingolipids and ceramides, sunscreens, such as, for example, octyl methoxycinnamate, 3-benzophenone or butylmethoxydibenzoylmethane, retinoic acid and its esters, and their mixtures.

13. The use as claimed in claim 9, characterized in that the active compound is chosen from α-hydroxy acids, such as lactic acid or glycolic acid, ascorbic acid and its biologically compatible salts, enzymes, components with a tightening effect, hydroxy acids and their salts, hydroxylated polyacids, sucrose and its derivatives, urea, amino acids, oligopeptides, water-soluble plant extracts, peptide or protein hydrolysates, bacterial ferments, trace elements, hyaluronic acid and its salts, mucopolysaccharides, vitamins B2, B6, H, PP, panthenol, folic acid, β-hydroxy acids, such as acetylsalicylic acid, allantoin, glycyrrhetic acid, kojic acid, hydroquinone, arginine, lysine, proline, serine, flavonoids, ginkgo biloba extracts, and their mixtures.

14. The use as claimed in claim 1, characterized in that the product is devoid of water-insoluble support layer.

15. A cosmetic product for making up and/or caring for the lips comprising an anhydrous film, this film being formed of at least one layer composed of a matrix based on at least one water-soluble polymer in which is included at least one coloring agent, said coloring agent being chosen from reflecting pigments, goniochromatic pigments, interference pigments, pearlescent pigments, fluorescent pigments, glitter and their mixtures.

16. A cosmetic product for making up and/or caring for the lips comprising:
an anhydrous film, this film being formed of at least one layer composed of a matrix based on at least one water-soluble polymer in which is included at least one coloring agent, and
an active compound chosen from compounds having a softening, healing, regenerating or soothing action or an action targeted at enhancing blood microcirculation in order to render the lips naturally more colored, in order to increase their volume or in order to smooth their surface.

17. A cosmetic product for making up and/or caring for the lips comprising:
an anhydrous film, this film being formed of at least one layer composed of a matrix based on at least one water-soluble polymer in which is included at least one coloring agent, and
at least one active compound chosen from polyunsaturated fatty acids, sphingolipids and ceramides, octyl methoxycinnamate, retinoic acid and its esters, and their mixtures.

18. A cosmetic product for making up and/or caring for the lips comprising:
an anhydrous film, this film being formed of at least one layer composed of a matrix based on at least one water-soluble polymer in which is included at least one coloring agent, and
at least one active compound chosen from glycolic acid, enzymes, components with a tightening effect, hydroxy acids and their salts, hydroxylated polyacids, amino acids, oligopeptides, peptide or protein hydrolysates, bacterial ferments, trace elements, hyaluronic acid and its salts, mucopolysaccharides, vitamins B2, B6, H, PP, panthenol, folic acid, β-hydroxy acids, such as acetylsalicylic acid, allantoin, glycyrrhetic acid, kojic acid, arginine, lysine, proline, serine, flavonoids, ginkgo biloba extracts, and their mixtures.

19. The cosmetic product as claimed in claim 15, characterized in that it is devoid of a water-insoluble support layer.

20. A method for making up the lips, characterized in that it comprises the facts of applying, to the lips, a cosmetic product as defined according to claim 15 and of dissolving the product by the action of the saliva and the rubbing of the lips against one another or the pressure of the finger.
